(19) United States
(12) Patent Application Publication
(10) Pub. No.: US 2025/0362312 A1
(43) Pub. Date: Nov. 27, 2025

(54) METHOD AND DEVICE FOR IDENTIFYING STRUCTURAL POLYMORPHISM OF FIBROUS PROTEIN OR PEPTIDE

(71) Applicant: HAMAMATSU PHOTONICS K.K., Hamamatsu-shi (JP)

(72) Inventors: Akinori Oda, Hamamatsu-shi (JP); Hiroshi Satozono, Hamamatsu-shi (JP); Tomoo Inubushi, Hamamatsu-shi (JP)

(73) Assignee: HAMAMATSU PHOTONICS K.K., Hamamatsu-shi (JP)

(21) Appl. No.: 19/192,656

(22) Filed: Apr. 29, 2025

(30) Foreign Application Priority Data
May 24, 2024 (JP) 2024-085152

(51) Int. Cl. G01N 33/68; G01N 21/64

(52) U.S. Cl. CPC G01N 33/6896; G01N 21/6408; G01N 2333/4709

(57) ABSTRACT

A method for identifying a structural polymorphism of a fibrous protein or peptide, the method including the steps of: obtaining a fluorescence decay curve for a sample containing a fibrous protein or peptide and thioflavin T; performing exponential fitting of four or more components based on a specific function G(t) on a function F(t) of the fluorescence decay curve to obtain fluorescence lifetime values τ₁ to τₙ and weighting factors A₁ to Aₙ (n is a natural number of 4 or more) of the respective exponential components; and identifying the structural polymorphism of the fibrous protein or peptide based on at least one or more values of the fluorescence lifetime values τ₁ to τₙ and at least one or more values of the weighting factors A₁ to Aₙ (a fluorescence lifetime value and a weighting factor in an exponential component derived from autofluorescence of thioflavin T are excluded).

### CROSS-REFERENCE TO RELATED APPLICATION

This application claims the benefit of Japanese patent application No. 2024-085152 filed on May 24, 2024, the disclosure of which is herein incorporated by reference in its entirety.

### TECHNICAL FIELD

The present invention relates to a method and a device for identifying the structural polymorphism of a fibrous protein or peptide.

### BACKGROUND

Amyloid is a fibrous abnormal aggregate formed by a change in the steric structure or properties or the like of a peptide or protein. Fibrous proteins such as amyloid have a diverse fibrous structure (structural polymorphism) even if the proteins forming the amyloid are the same. It is known that when proteins in the body form amyloid and the amyloid accumulates in the organs, the amyloid causes various diseases, and it is also known that diseases caused by the structural polymorphism of the amyloid are different.

For example, Fitzpatrick AWP et al., “Cryo-EM structures of tau filaments from Alzheimer's disease.”, Nature. 2017 Jul. 13; 547 (7662): 185-190, Falcon B, et al. “Structures of filaments from Pick's disease reveal a novel tau protein fold.” Nature. 2018 September; 561 (7721): 137-140, and Falcon B, et al., “Novel tau filament fold in chronic traumatic encephalopathy encloses hydrophobic molecules.” Nature. 2019 April; 568 (7752): 420-423 describe that the structures of tau amyloid are different among patients with Alzheimer's disease, Pick's disease, and chronic traumatic encephalopathy. For example, Schweighauser M, et al., “Structures of α-synuclein filaments from multiple system atrophy.” Nature. 2020 September; 585 (7825): 464-469 and Yang Y, et al., “Structures of α-synuclein filaments from human brains with Lewy pathology.” Nature. 2022 October; 610 (7933): 791-795 describe that the structures of α-synuclein amyloid are different among patients with multiple system atrophy, dementia with Lewy bodies, and Parkinson's disease.

### SUMMARY

### Technical Problem

As described above, attention has been paid to identifying the structural polymorphism of a fibrous protein such as amyloid. Here, in Fitzpatrick AWP et al., “Cryo-EM structures of tau filaments from Alzheimer's disease.”, Nature. 2017 Jul. 13; 547 (7662): 185-190, Falcon B, et al. “Structures of filaments from Pick's disease reveal a novel tau protein fold.” Nature. 2018 September; 561 (7721): 137-140, Falcon B, et al., “Novel tau filament fold in chronic traumatic encephalopathy encloses hydrophobic molecules.” Nature. 2019 April; 568 (7752): 420-423, Schweighauser M, et al., “Structures of α-synuclein filaments from multiple system atrophy.” Nature. 2020 September; 585 (7825): 464-469, and Yang Y, et al., “Structures of α-synuclein filaments from human brains with Lewy pathology.” Nature. 2022 October; 610 (7933): 791-795, a cryo-electron microscope is used to identify the structural polymorphism of amyloid. Meanwhile, in the case of identifying the structural polymorphism of amyloid using the cryo-electron microscope, the cost is high and the measurement time is also long (about 1 to 2 months). The structural polymorphism of amyloid can be identified using a solid state nuclear magnetic resonance method, but also in this case, the cost is high and the measurement time is long (about several weeks).

An object of the present invention is to provide a method capable of easily and quickly identifying the structural polymorphism of a fibrous protein or peptide. It is another object of the present invention to provide a device and a program that can be used for the method.

### Solution to Problem

The present inventors separated a fluorescence decay curve for thioflavin T (hereinafter, also referred to as “ThT”) bound to amyloid β fibers into four exponential components, and found that the fluorescence lifetime values of the three components were derived from ThT bound (in a binding manner) to different binding sites of the amyloid β fibers, respectively. The present inventors found that weighting factors in the exponential components indicate the abundances of different binding sites assumed from the fluorescence lifetime values. From this, it is considered that by measuring the fluorescence lifetime value for each binding site and measuring the weighting factor for each abundance of the binding site, a slight difference in the structure of the fibrous protein or peptide can be measured as differences between the fluorescence lifetime values and the weighting factors.

The present invention relates to a method for identifying a structural polymorphism of a fibrous protein or peptide, the method including the steps of:
obtaining a fluorescence decay curve for a sample containing a fibrous protein or peptide and thioflavin T;
performing exponential fitting of four or more components based on a function G(t) represented by the following Mathematical Formula (1) on a function F(t) of the fluorescence decay curve to obtain fluorescence lifetime values τ₁ to τₙ and weighting factors A₁ to Aₙ (n is a natural number of 4 or more) of the respective exponential components; and
identifying the structural polymorphism of the fibrous protein or peptide based on at least one or more values of the fluorescence lifetime values τ₁ to τₙ and at least one or more values of the weighting factors A₁ to Aₙ (a fluorescence lifetime value and a weighting factor in an exponential component derived from autofluorescence of thioflavin T are excluded).

$$\begin{matrix}[ Math . 1 ] & \\ G ( t ) = {\sum }_{í = 1}^{n} [ {A}_{i} exp ( - t / {τ}_{i} ) ] & ( 1 )\end{matrix}$$

[In Mathematical Formula (1), n represents a natural number of 4 or more, Aᵢ represents the weighting factor of each exponential component, t represents a variable representing a time, and τᵢ represents the fluorescence lifetime value of each exponential component.]

Since the method for identifying a structural polymorphism of a fibrous protein or peptide according to the present invention includes the above configuration, it is possible to easily and quickly identify the structural polymorphism of the fibrous protein or peptide.

In the method for identifying a structural polymorphism of a fibrous protein or peptide, the exponential fitting may include: convolution-integrating the function G(t) according to the following Mathematical Formula (2) to obtain a function I(t); and comparing the function I(t) with the function F(t) of the fluorescence decay curve and searching a combination of variables that minimizes χ² in the following Mathematical Formula (3) by a nonlinear least squares method. In that case, χ² is desirably 1.2 or less.

$$\begin{matrix}[ Math . 2 ] & \\ I ( t ) = \int E ( {t}^{′} ) G ( t - {t}^{′} ) {dt}^{′} + C = {\sum }_{{t}^{′} = 0}^{t} [ E ( {t}^{′} ) G ( t - {t}^{′} ) ] + C & ( 2 )\end{matrix}$$

[In Mathematical Formula (2), E(t) represents a device response function of a fluorescence lifetime measuring device, C represents a background, and t and t′ represent variables representing a time.]

$$\begin{matrix}[ Math . 3 ] & \\ {χ}^{2} = {\sum }_{j = {p}_{1}}^{{p}_{2}} \frac{{[ I ( {t}_{j} ) - F ( {t}_{j} ) ]}^{2}}{I ( {t}_{j} )} / ( {p}_{2} - {p}_{1} + 1 ) & ( 3 )\end{matrix}$$

[In Mathematical Formula (3), tⱼ represents a variable representing a time, p₁ represents a start time of analysis, and p₂ represents an end time of analysis.]

In the method for identifying a structural polymorphism of a fibrous protein or peptide, the identifying step may be performed by collating the fluorescence lifetime values τ₁ to τₙ and the weighting factors A₁ to Aₙ with a database storing fluorescence lifetime values τ₁ to τₙ and weighting factors A₁ to Aₙ associated with different structural polymorphisms of the fibrous protein or peptide, respectively. This makes it possible to estimate the structural polymorphism of the fibrous protein or peptide.

In the method for identifying a structural polymorphism of a fibrous protein or peptide, the exponential fitting may be exponential fitting of four components.

In the method for identifying a structural polymorphism of a fibrous protein or peptide, the protein or peptide that forms the fibrous protein or peptide may be an amyloid-forming protein or peptide.

In the method for identifying a structural polymorphism of a fibrous protein or peptide, the amyloid-forming protein or peptide may be one or more selected from the group consisting of amyloid β, a synuclein, transactive response DNA-binding protein-43, superoxide dismutase 1, prion protein, β2 microglobulin, immunoglobulin light chain protein, transthyretin, tau, and partial peptides thereof.

The present invention also relates to a device for identifying a structural polymorphism of a fibrous protein or peptide, the device including:
a fluorescence lifetime measurement unit configured to obtain a fluorescence decay curve for a sample containing a fibrous protein or peptide and thioflavin T;
a first calculation unit configured to perform exponential fitting of four or more components based on a function G(t) represented by the following Mathematical Formula (1) on the fluorescence decay curve to obtain fluorescence lifetime values τ₁ to τₙ and weighting factors A₁ to Aₙ (n is a natural number of 4 or more) of the respective exponential components; and
a second calculation unit configured to identify the structural polymorphism of the fibrous protein or peptide based on at least one or more values of the fluorescence lifetime values τ₁ to τₙ and at least one or more values of the weighting factors A₁ to Aₙ (a fluorescence lifetime value and a weighting factor in an exponential component derived from autofluorescence of thioflavin T are excluded).

$$\begin{matrix}[ Math . 4 ] & \\ G ( t ) = {\sum }_{í = 1}^{n} [ {A}_{i} exp ( - t / {τ}_{i} ) ] & ( 1 )\end{matrix}$$

[In Mathematical Formula (1), n represents a natural number of 4 or more, Aᵢ represents the weighting factor of each exponential component, t represents a variable representing a time, and τᵢ represents the fluorescence lifetime value of each exponential component.]

In the device for identifying the structural polymorphism of a fibrous protein or peptide, the exponential fitting may include: convolution-integrating the function G(t) according to the following Mathematical Formula (2) to obtain a function I(t); and comparing the function I(t) with the function F(t) of the fluorescence decay curve and searching a combination of variables that minimizes χ² in the following Mathematical Formula (3) by a nonlinear least squares method. In that case, χ² is desirably 1.2 or less.

$$\begin{matrix}[ Math . 5 ] & \\ I ( t ) = \int E ( {t}^{′} ) G ( t - {t}^{′} ) {dt}^{′} + C = {\sum }_{{t}^{′} = 0}^{t} [ E ( {t}^{′} ) G ( t - {t}^{′} ) ] + C & ( 2 )\end{matrix}$$

[In Mathematical Formula (2), E(t) represents a device response function of a fluorescence lifetime measuring device, C represents a background, and t and t′ represent variables representing a time.]

$$\begin{matrix}[ Math . 6 ] & \\ {χ}^{2} = {\sum }_{j = {p}_{1}}^{{p}_{2}} \frac{{[ I ( {t}_{j} ) - F ( {t}_{j} ) ]}^{2}}{I ( {t}_{j} )} / ( {p}_{2} - {p}_{1} + 1 ) & ( 3 )\end{matrix}$$

[In Mathematical Formula (3), tⱼ represents a variable representing a time, p₁ represents a start time of analysis, and p₂ represents an end time of analysis.]

In the device for identifying the structural polymorphism of a fibrous protein or peptide, the identifying may be performed by collating the fluorescence lifetime values τ₁ to τₙ and the weighting factors A₁ to Aₙ with a database storing fluorescence lifetime values τ₁ to τₙ and weighting factors A₁ to Aₙ associated with different structural polymorphisms of the fibrous protein or peptide, respectively.

In the device for identifying the structural polymorphism of a fibrous protein or peptide, the exponential fitting may be exponential fitting of four components.

In the device for identifying the structural polymorphism of a fibrous protein or peptide, a protein or peptide that forms the fibrous protein or peptide may be an amyloid-forming protein or peptide.

In the device for identifying the structural polymorphism of a fibrous protein or peptide, the amyloid-forming protein or peptide may be one or more selected from the group consisting of amyloid β, a synuclein, transactive response DNA-binding protein-43, superoxide dismutase 1, prion protein, β2 microglobulin, immunoglobulin light chain protein, transthyretin, tau, and partial peptides thereof.

The present invention also relates to a program for identifying a structural polymorphism of a fibrous protein or peptide, the program for causing a computer to function as: a fluorescence lifetime measurement unit configured to obtain a fluorescence decay curve for a sample containing a fibrous protein or peptide and thioflavin T; a first calculation unit configured to perform exponential fitting of four or more components based on a function G(t) represented by the following Mathematical Formula (1) on a function F(t) of the fluorescence decay curve to obtain at least one or more values of fluorescence lifetime values τ₁ to τₙ and at least one or more values of weighting factors A₁ to Aₙ (n is a natural number of 4 or more) of the respective exponential components; and a second calculation unit configured to identify the structural polymorphism of the fibrous protein or peptide based on the fluorescence lifetime values τ₁ to τₙ and the weighting factors A₁ to Aₙ (a fluorescence lifetime value and a weighting factor in an exponential component derived from autofluorescence of thioflavin T are excluded).

$$\begin{matrix}[ Math . 7 ] & \\ G ( t ) = {\sum }_{í = 1}^{n} [ {A}_{i} exp ( - t / {τ}_{i} ) ] & ( 1 )\end{matrix}$$

[In Mathematical Formula (1), n represents a natural number of 4 or more, Aᵢ represents the weighting factor of each exponential component, t represents a variable representing a time, and τᵢ represents the fluorescence lifetime value of each exponential component.]

In the program for identifying a structural polymorphism of a fibrous protein or peptide, the exponential fitting may include: convolution-integrating the function G(t) according to the following Mathematical Formula (2) to obtain a function I(t); and comparing the function I(t) with the function F(t) of the fluorescence decay curve and searching a combination of variables that minimizes χ² in the following Mathematical Formula (3) by a nonlinear least squares method. In that case, χ² is desirably 1.2 or less.

$$[ Math . 8 ]$$

$$I ( t ) = \int E ( {t}^{′} ) G ( t - {t}^{′} ) {dt}^{′} + C = {\sum }_{{t}^{′} = 0}^{t} [ E ( {t}^{′} ) G ( t - {t}^{′} ) ] + C \qquad ( 2 )$$

[In Mathematical Formula (2), E(t) represents a device response function of a fluorescence lifetime measuring device, C represents a background, and t and t′ represent variables representing a time.]

$$[ Math . 9 ]$$

$${χ}^{2} = {\sum }_{j = {p}_{1}}^{{p}_{2}} \frac{{[ I ( {t}_{j} ) - F ( {t}_{j} ) ]}^{2}}{I ( {t}_{j} )} / ( {p}_{2} - {p}_{1} + 1 ) \qquad ( 3 )$$

[In Mathematical Formula (3), tⱼ represents a variable representing a time, p₁ represents a start time of analysis, and p₂ represents an end time of analysis.]

In the program for identifying a structural polymorphism of a fibrous protein or peptide, the identifying may be performed by collating the fluorescence lifetime values τ₁ to τₙ and the weighting factors A₁ to Aₙ with a database storing fluorescence lifetime values τ₁ to τₙ and weighting factors A₁ to Aₙ associated with different structural polymorphisms of the fibrous protein or peptide, respectively.

In the program for identifying a structural polymorphism of a fibrous protein or peptide, the exponential fitting may be exponential fitting of four components.

In the program for identifying a structural polymorphism of a fibrous protein or peptide, the protein or peptide that forms the fibrous protein or peptide may be an amyloid-forming protein or peptide.

In the program for identifying a structural polymorphism of a fibrous protein or peptide, the amyloid-forming protein or peptide may be one or more selected from the group consisting of amyloid β, a synuclein, transactive response DNA-binding protein-43, superoxide dismutase 1, prion protein, β2 microglobulin, immunoglobulin light chain protein, transthyretin, tau, and partial peptides thereof.

The present invention includes, for example, the following inventions.

[1]

A method for identifying a structural polymorphism of a fibrous protein or peptide, the method including the steps of:
obtaining a fluorescence decay curve for a sample comprising a fibrous protein or peptide and thioflavin T;
performing exponential fitting of four or more components based on a function G(t) represented by the following Mathematical Formula (1) on a function F(t) of the fluorescence decay curve to obtain fluorescence lifetime values τ₁ to τₙ and weighting factors A₁ to Aₙ (n is a natural number of 4 or more) of the respective exponential components; and
identifying the structural polymorphism of the fibrous protein or peptide based on at least one or more values of the fluorescence lifetime values τ₁ to τₙ and at least one or more values of the weighting factors A₁ to Aₙ (a fluorescence lifetime value and a weighting factor in an exponential component derived from autofluorescence of thioflavin T are excluded).

$$[ Math . 10 ]$$

$$G ( t ) = {\sum }_{í = 1}^{n} [ {A}_{i} exp ( - t / {τ}_{i} ) ] \qquad ( 1 )$$

[In Mathematical Formula (1), n represents a natural number of 4 or more, Aᵢ represents the weighting factor of each exponential component, t represents a variable representing a time, and τᵢ represents the fluorescence lifetime value of each exponential component.]
   [2]

The method according to [1], wherein the exponential fitting includes: convolution-integrating the function G(t) according to the following Mathematical Formula (2) to obtain a function I(t); and
comparing the function I(t) with the function F(t) of the fluorescence decay curve and searching a combination of variables that minimizes χ² in the following Mathematical Formula (3) by a nonlinear least squares method.

$$[ Math . 11 ]$$

$$I ( t ) = \int E ( {t}^{′} ) G ( t - {t}^{′} ) {dt}^{′} + C = {\sum }_{{t}^{′} = 0}^{t} [ E ( {t}^{′} ) G ( t - {t}^{′} ) ] + C \qquad ( 2 )$$

[In Mathematical Formula (2), E(t) represents a device response function of a fluorescence lifetime measuring device, C represents a background, and t and t′ represent variables representing a time.]

$$[ Math . 12 ]$$

$${χ}^{2} = {\sum }_{j = {p}_{1}}^{{p}_{2}} \frac{{[ I ( {t}_{j} ) - F ( {t}_{j} ) ]}^{2}}{I ( {t}_{j} )} / ( {p}_{2} - {p}_{1} + 1 ) \qquad ( 3 )$$

[In Mathematical Formula (3), tⱼ represents a variable representing a time, p₁ represents a start time of analysis, and p₂ represents an end time of analysis.]
   [3]

The method according to [1] or [2], wherein the identifying step is performed by collating the fluorescence lifetime values τ₁ to τₙ and the weighting factors A₁ to Aₙ with a database storing fluorescence lifetime values τ₁ to τₙ and weighting factors A₁ to Aₙ associated with different structural polymorphisms of the fibrous protein or peptide, respectively.

[4]

The method according to any one of [1] to [3], wherein the exponential fitting is exponential fitting of four components.

[5]

The method according to any one of [1] to [4], wherein a protein or peptide that forms the fibrous protein or peptide is an amyloid-forming protein or peptide.

[6]

The method according to [5], wherein the amyloid-forming protein or peptide is one or more selected from the group consisting of amyloid β, α synuclein, transactive response DNA-binding protein-43, superoxide dismutase 1, prion protein, β2 microglobulin, immunoglobulin light chain protein, transthyretin, tau, and partial peptides thereof.

[7]

A device for identifying a structural polymorphism of a fibrous protein or peptide, the device including:
a fluorescence lifetime measurement unit configured to obtain a fluorescence decay curve for a sample comprising a fibrous protein or peptide and thioflavin T;
a first calculation unit configured to perform exponential fitting of four or more components based on a function G(t) represented by the following Mathematical Formula (1) on a function F(t) of the fluorescence decay curve to obtain fluorescence lifetime values τ₁ to τₙ and weighting factors A₁ to Aₙ (n is a natural number of 4 or more) of the respective exponential components; and
a second calculation unit configured to identify the structural polymorphism of the fibrous protein or peptide based on at least one or more values of the fluorescence lifetime values τ₁ to τₙ and at least one or more values of the weighting factors A₁ to Aₙ (a fluorescence lifetime value and a weighting factor in an exponential component derived from autofluorescence of thioflavin T are excluded).

$$[ Math . 13 ]$$

$$G ( t ) = {\sum }_{í = 1}^{n} [ {A}_{i} exp ( - t / {τ}_{i} ) ] \qquad ( 1 )$$

[In Mathematical Formula (1), n represents a natural number of 4 or more, Aᵢ represents the weighting factor of each exponential component, t represents a variable representing a time, and τᵢ represents the fluorescence lifetime value of each exponential component.]
   [8]

The device according to [7], wherein the exponential fitting includes: convolution-integrating the function G(t) according to the following Mathematical Formula (2) to obtain a function I(t); and
comparing the function I(t) with the function F(t) of the fluorescence decay curve and searching a combination of variables that minimizes χ² in the following Mathematical Formula (3) by a nonlinear least squares method.

$$[ Math . 14 ]$$

$$I ( t ) = \int E ( {t}^{′} ) G ( t - {t}^{′} ) {dt}^{′} + C = {\sum }_{{t}^{′} = 0}^{t} [ E ( {t}^{′} ) G ( t - {t}^{′} ) ] + C \qquad ( 2 )$$

[In Mathematical Formula (2), E(t) represents a device response function of a fluorescence lifetime measuring device, C represents a background, and t and t′ represent variables representing a time.]

$$[ Math . 15 ]$$

$${χ}^{2} = {\sum }_{j = {p}_{1}}^{{p}_{2}} \frac{{[ I ( {t}_{j} ) - F ( {t}_{j} ) ]}^{2}}{I ( {t}_{j} )} / ( {p}_{2} - {p}_{1} + 1 ) \qquad ( 3 )$$

[In Mathematical Formula (3), tⱼ represents a variable representing a time, p₁ represents a start time of analysis, and p₂ represents an end time of analysis.]
   [9]

The device according to [7] or [8], wherein the identifying is performed by collating the fluorescence lifetime values τ₁ to τₙ and the weighting factors A₁ to Aₙ with a database storing fluorescence lifetime values τ₁ to τₙ and weighting factors A₁ to Aₙ associated with different structural polymorphisms of the fibrous protein or peptide, respectively.

[10]

The device according to any one of [7] to [9], wherein the exponential fitting is exponential fitting of four components.

[11]

The device according to any one of [7] to [10], wherein a protein or peptide that forms the fibrous protein or peptide is an amyloid-forming protein or peptide.

[12]

The device according to [11], wherein the amyloid-forming protein or peptide is one or more selected from the group consisting of amyloid β, α synuclein, transactive response DNA-binding protein-43, superoxide dismutase 1, prion protein, β2 microglobulin, immunoglobulin light chain protein, transthyretin, tau, and partial peptides thereof.

[13]

A program for identifying a structural polymorphism of a fibrous protein or peptide, the program for causing a computer to function as:
a fluorescence lifetime measurement unit configured to obtain a fluorescence decay curve for a sample comprising a fibrous protein or peptide and thioflavin T;
a first calculation unit configured to perform exponential fitting of four or more components based on a function G(t) represented by the following Mathematical Formula (1) on a function F(t) of the fluorescence decay curve to obtain fluorescence lifetime values τ₁ to τₙ and weighting factors A₁ to Aₙ (n is a natural number of 4 or more) of the respective exponential components; and
a second calculation unit configured to identify the structural polymorphism of the fibrous protein or peptide based on at least one or more values of the fluorescence lifetime values τ₁ to τₙ and at least one or more values of the weighting factors A₁ to Aₙ (a fluorescence lifetime value and a weighting factor in an exponential component derived from autofluorescence of thioflavin T are excluded).

$$[ Math . 16 ]$$

$$G ( t ) = {\sum }_{í = 1}^{n} [ {A}_{i} exp ( - t / {τ}_{i} ) ] \qquad ( 1 )$$

[In Mathematical Formula (1), n represents a natural number of 4 or more, Aᵢ represents the weighting factor of each exponential component, t represents a variable representing a time, and τᵢ represents the fluorescence lifetime value of each exponential component.]
   [14]

The program according to [13], wherein the exponential fitting includes: convolution-integrating the function G(t) according to the following Mathematical Formula (2) to obtain a function I(t); and
comparing the function I(t) with the function F(t) of the fluorescence decay curve and searching a combination of variables that minimizes χ² in the following Mathematical Formula (3) by a nonlinear least squares method.

$$[ Math . 17 ]$$

$$I ( t ) = \int E ( {t}^{′} ) G ( t - {t}^{′} ) {dt}^{′} + C = {\sum }_{{t}^{′} = 0}^{t} [ E ( {t}^{′} ) G ( t - {t}^{′} ) ] + C \qquad ( 2 )$$

[In Mathematical Formula (2), E(t) represents a device response function of a fluorescence lifetime measuring device, C represents a background, and t and t′ represent variables representing a time.]

$$[ Math . 18 ]$$

$${χ}^{2} = {\sum }_{j = {p}_{1}}^{{p}_{2}} \frac{{[ I ( {t}_{j} ) - F ( {t}_{j} ) ]}^{2}}{I ( {t}_{j} )} / ( {p}_{2} - {p}_{1} + 1 ) \qquad ( 3 )$$

[In Mathematical Formula (3), tⱼ represents a variable representing a time, p₁ represents a start time of analysis, and p₂ represents an end time of analysis.]
   [15]

The program according to or [14], wherein the identifying is performed by collating the fluorescence lifetime values τ₁ to τₙ and the weighting factors A₁ to Aₙ with a database storing fluorescence lifetime values τ₁ to τₙ and weighting factors A₁ to Aₙ associated with different structural polymorphisms of the fibrous protein or peptide, respectively.

[16]

The program according to any one of to [15], wherein the exponential fitting is exponential fitting of four components.

[17]

The program according to any one of to [16], wherein a protein or peptide that forms the fibrous protein or peptide is an amyloid-forming protein or peptide.

[18]

The program according to [17], wherein the amyloid-forming protein or peptide is one or more selected from the group consisting of amyloid β, α synuclein, transactive response DNA-binding protein-43, superoxide dismutase 1, prion protein, β2 microglobulin, immunoglobulin light chain protein, transthyretin, tau, and partial peptides thereof.

### Advantageous Effects of Invention

The present invention can provide a method capable of easily and quickly identifying a structural polymorphism of a fibrous protein or peptide. The present invention can also provide a device and a program that can be used for the method.

### BRIEF DESCRIPTION OF THE DRAWINGS

FIG. 1 is a schematic diagram showing the hardware configuration of a device for identifying the structural polymorphism of a fibrous protein or peptide;

FIG. 2 is a schematic diagram showing the functional configuration of the device for identifying the structural polymorphism of a fibrous protein or peptide;

FIG. 3 is a fluorescence decay curve for a sample containing amyloid β fibers and ThT;

FIG. 4A is a graph in which the fluorescence intensity of each exponential component obtained in a case where amyloid β fibers are used as an analysis object when a measurement sample is prepared with the concentration of amyloid β set to 2 μM is plotted against the molar concentration ratio of the amyloid β fibers and ThT ([ThT]/[amyloid β]), and FIG. 4B is a graph in which the fluorescence intensity of each exponential component obtained in a case where amyloid β fibers are used as an analysis object when a measurement sample is prepared with the concentration of amyloid β set to 0.8 μM is plotted against the molar concentration ratio of the amyloid β fibers and ThT ([ThT]/[amyloid β]);

FIG. 5A is a graph showing the comparison result of amyloid β (1-40) fibers and amyloid β (1-42) fibers with respect to a fluorescence lifetime value in an exponential component C3, and FIG. 5B is a graph showing the comparison result of amyloid β (1-40) fibers and amyloid β (1-42) fibers with respect to a fluorescence lifetime value in an exponential component C4; and

FIG. 6A is a graph showing the comparison result of amyloid β (1-42) fibers formed under different conditions with respect to the fluorescence lifetime value in the exponential component C3 and FIG. 6B is a graph showing the comparison result of amyloid β (1-42) fibers formed under different conditions with respect to the fluorescence lifetime value in the exponential component C4.

### DETAILED DESCRIPTION

Hereinafter, embodiments of the present invention will be described in detail. However, the present invention is not limited to the following embodiments.

### [Method for Identifying Structural Polymorphism of Fibrous Protein or Peptide]

A method for identifying a structural polymorphism of a fibrous protein or peptide according to the present embodiment (hereinafter, also referred to as “identifying method according to the present embodiment”) includes the steps of: obtaining a fluorescence decay curve for a sample containing the fibrous protein or peptide and ThT (measuring step); performing exponential fitting of four or more components based on a function G(t) represented by the following Mathematical Formula (1) on a function F(t) of the fluorescence decay curve to obtain fluorescence lifetime values τ₁ to τₙ and weighting factors A₁ to Aₙ (n is a natural number of 4 or more) of the respective exponential components (fitting step); and identifying the structural polymorphism of the fibrous protein or peptide based on at least one or more values of the fluorescence lifetime values τ₁ to τₙ and at least one or more values of the weighting factors A₁ to Aₙ (a fluorescence lifetime value and a weighting factor in an exponential component derived from autofluorescence of ThT are excluded) (identifying step).

$$[ Math . 19 ]$$

$$G ( t ) = {\sum }_{í = 1}^{n} [ {A}_{i} exp ( - t / {τ}_{i} ) ] \qquad ( 1 )$$

[In Mathematical Formula (1), n represents a natural number of 4 or more, Aᵢ represents the weighting factor of each exponential component, t represents a variable representing a time, and t; represents the fluorescence lifetime value of each exponential component.]

In the present specification, the “structural polymorphism of a fibrous protein or peptide” means that a fibrous protein or peptide formed by one peptide or protein (peptide or protein having the same amino acid sequence) has a different structure.

### (Measuring Step)

The measuring step is a step of obtaining a fluorescence decay curve for a sample containing a fibrous protein or peptide and ThT. In the measuring step, the sample containing a fibrous protein or peptide and ThT is irradiated with excitation light that excites ThT, and the fluorescence decay of the sample containing a fibrous protein or peptide and ThT is measured.

The sample containing a fibrous protein or peptide and ThT is not particularly limited as long as these are contained in the sample, and can be prepared by mixing a sample containing a fibrous protein or peptide with ThT. The sample containing a fibrous protein or peptide may or may not be a biological sample. Examples of the biological sample containing a fibrous protein or peptide include a brain section, cerebrospinal fluid, blood, and a mucous membrane. The sample containing a fibrous protein or peptide may be a solution obtained by suspending or dissolving these samples in an appropriate medium (for example, water, buffer solution, medium, and the like), or may be a solution obtained by filtering an insoluble matter after suspending or dissolving the samples. The sample containing a fibrous protein or peptide and ThT may be a liquid or a solid, but is preferably a liquid.

In the present specification, the “fibrous protein or peptide” is a protein or peptide aggregate formed by the assembly of protein or peptide molecules in fibrous forms. The fibrous protein or peptide may be, for example, amyloid. The “amyloid” means a special insoluble aggregate (fibrous protein or peptide) of a protein or peptide characterized by a β-sheet structure. The protein or peptide that forms the fibrous protein or peptide is not particularly limited, and may be, for example, an amyloid-forming protein or peptide. The amyloid-forming protein or peptide is, for example, preferably one or more selected from the group consisting of amyloid β, α synuclein, transactive response DNA-binding protein-43, superoxide dismutase 1, prion protein, β2 microglobulin, immunoglobulin light chain protein, transthyretin, tau, and partial peptides thereof, and preferably one or more selected from the group consisting of amyloid β, α synuclein, tau, and partial peptides thereof. The protein or peptide that forms the fibrous protein or peptide may be, for example, collagen, keratin, elastin, fibroin, actin, myosin, fibronectin, laminin, and the like.

Thioflavin T (ThT) is a known compound represented by the following formula and also referred to as 4-(3,6-dimethyl-1,3-benzothiazole-3-ium-2-yl)-N,N-dimethylaniline chloride. ThT is a fluorescent dye widely used for amyloid staining (ThT staining).

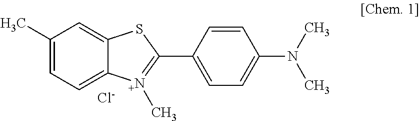

In the identifying method according to the present embodiment, for example, a commercially available ThT reagent can be used without particular limitation.

The concentration of the fibrous protein or peptide in the sample containing the fibrous protein or peptide and ThT, for example, when the sample is a liquid, may be 0.2 μM or more, 0.5 μM or more, 0.8 μM or more, 1 μM or more, 2 μM or more, 3 μM or more, 4 μM or more, or 5 μM or more, and may be 20 μM or less, 15 μM or less, or 10 μM or less. The concentration of ThT in the sample containing the fibrous protein or peptide and ThT, for example, when the sample is a solid, may be 0.2 μg/g or more, 0.4 μg/g or more, 0.6 μg/g or more, 0.8 μg/g or more, 1 μg/g or more, or 1.5 μg/g or more, and may be 10 μg/g or less, 9 μg/g or less, 8 μg/g or less, 7 μg/g or less, 6 μg/g or less, 5 μg/g or less, 4 μg/g or less, or 3 μg/g or less.

The concentration of ThT in the sample containing the fibrous protein or peptide and ThT, for example, when the sample is a liquid, may be 1 μM or more, 2 μM or more, 3 μM or more, 4 μM or more, or 5 UM or more, and may be 20 μM or less, 15 μM or less, or 10 μM or less. The concentration of ThT in the sample containing the fibrous protein or peptide and ThT, for example, when the sample is a solid, may be 0.2 μg/g or more, 0.4 μg/g or more, 0.6 μg/g or more, 0.8 μg/g or more, 1 μg/g or more, or 1.5 μg/g or more, and may be 10 μg/g or less, 9 μg/g or less, 8 μg/g or less, 7 μg/g or less, 6 μg/g or less, 5 μg/g or less, 4 μg/g or less, or 3 μg/g or less.

A ratio between the molar concentration of ThT and the molar concentration of the protein or peptide that forms the fibrous protein or peptide in the sample containing the fibrous protein or fibrous peptide and ThT ([ThT]/[protein or peptide that forms fibrous protein or peptide]) may be, for example, more than 0, 0.01 or more, 0.05 or more, 0.1 or more, 0.5 or more, 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, or 25 or more. The ratio between the molar concentration of ThT and the molar concentration of the protein or peptide that forms the fibrous protein or peptide in the sample containing the fibrous protein or fibrous peptide and ThT ([ThT]/[protein or peptide that forms fibrous protein or peptide]) may be, for example, 100 or less, 95 or less, 90 or less, 85 or less, 80 or less, 75 or less, 70 or less, 65 or less, 60 or less, 55 or less, 50 or less, 40 or less, 35 or less, or 30 or less. The molar concentration of the protein or peptide that forms the fibrous protein or peptide is a molar concentration converted to a protein or peptide (for example, amyloid β or the like) capable of forming the fibrous protein or peptide.

The fluorescence decay of the sample containing a fibrous protein or peptide and ThT can be measured by a known method. Specifically, for example, the fluorescence decay curve can be obtained by irradiating a sample containing a fibrous protein or peptide and ThT with light having a wavelength of 280 nm or more and 800 nm or less, 300 nm or more and 600 nm or less, 350 nm or more and 500 nm or less, or 400 nm or more and 420 nm or less (preferably 412 nm) and detecting fluorescence emitted from the sample and having a wavelength of 400 nm or more and 600 nm or less, 420 nm or more and 580 nm or less, or 450 nm or more and 550 nm or less over time using a fluorescence lifetime measuring device.

### (Fitting Step)

The fitting step is a step of performing exponential fitting of four or more components based on a function G(t) represented by the following Mathematical Formula (1) on a function F(t) of the fluorescence decay curve obtained in the measuring step to obtain fluorescence lifetime values τ₁ to τₙ and weighting factors A₁ to Aₙ (n is a natural number of 4 or more) of the respective exponential components.

$$[ Math . 20 ]$$

$$G ( t ) = {\sum }_{í = 1}^{n} [ {A}_{i} exp ( - t / {τ}_{i} ) ] \qquad ( 1 )$$

[In Mathematical Formula (1), n represents a natural number of 4 or more, Aᵢ represents the weighting factor of each exponential component, t represents a variable representing a time, and τᵢ represents the fluorescence lifetime value of each exponential component.]

The fluorescence expression of ThT bound to the fibrous protein or peptide is considered to be affected by the binding of ThT to the fibrous protein or peptide, thereby constraining the free rotation of a benzothiazole ring relative to an aminobenzene ring in ThT (Vitali I. et al., J. Phys. Chem. B 2008, 112, 49, 15893-15902). It is considered that the strength of binding between ThT and the fibrous protein or peptide affects the restraint of the free rotation. Therefore, it is considered that the fluorescence lifetime value of ThT of each exponential component reflects the strength of binding between ThT and the fibrous protein or peptide (that is, a difference in a binding site (binding manner) to the fibrous protein or peptide).

From the above, it is considered that the fluorescence lifetime values τ₁ to τₙ of each exponential component obtained by the fitting step reflect the difference in the binding site of ThT of the fibrous protein or peptide except for a fluorescence lifetime value in an exponential component derived from autofluorescence of ThT. It is considered that the weighting factor of each exponential component reflects the abundances of different binding sites corresponding to the fluorescence lifetime value except for a weighting factor in an exponential component derived from autofluorescence of ThT. Therefore, based on these values, the structural polymorphism of the fibrous protein or peptide can be identified in the identifying step described later. The fluorescence lifetime value in an exponential component derived from autofluorescence of ThT is a fluorescence lifetime value derived from ThT not bound to the fibrous protein or peptide, and is the smallest among the obtained fluorescence lifetime values τ₁ to τₙ. The weighting factor in an exponential component derived from autofluorescence of ThT is combined with the fluorescence lifetime value in an exponential component derived from autofluorescence of ThT.

For example, the fitting step may include: convolution-integrating the function G(t) according to the following Mathematical Formula (2) to obtain a function I(t); and comparing the function I(t) with the function F(t) of the fluorescence decay curve and searching a combination of variables (τ₁ to τₙ, Aᵢ to Aₙ) that minimizes χ² in the following Mathematical Formula (3) by a nonlinear least squares method. By performing the above search, the best combination of τ₁ to τₙ and A₁ to Aₙ in Mathematical Formula (1) is obtained.

τ₁ to τₙ obtained as a result of this analysis are the lifetime values of the respective fluorescent components, and A₁ to Aₙ represent the weighting factors of the respective fluorescent components, that is, the amounts of the fluorescent components.

$$[ Math . 21 ]$$

$$I ( t ) = \int E ( {t}^{′} ) G ( t - {t}^{′} ) {dt}^{′} + C = {\sum }_{{t}^{′} = 0}^{t} [ E ( {t}^{′} ) G ( t - {t}^{′} ) ] + C \qquad ( 2 )$$

[In Mathematical Formula (2), E(t) represents a device response function of a fluorescence lifetime measuring device, C represents a background, and t and t′ represent variables representing a time.]

$$[ Math . 22 ]$$

$${χ}^{2} = {\sum }_{j = {p}_{1}}^{{p}_{2}} \frac{{[ I ( {t}_{j} ) - F ( {t}_{j} ) ]}^{2}}{I ( {t}_{j} )} / ( {p}_{2} - {p}_{1} + 1 ) \qquad ( 3 )$$

[In Mathematical Formula (3), tⱼ represents a variable representing a time, p₁ represents a start time of analysis, and p₂ represents an end time of analysis.]

Here, the value of n is the number of components representing the number of exponential functions necessary for the analysis of the fluorescence decay curve, and each component originates from a different physical mechanism and/or fluorescence component. The value of n may be any value, but is preferably a value sufficient to obtain good fitting with χ²≤1.2 as a guide, and more preferably 4 components. In the present embodiment, these components originate from a fluorescent component derived from a complex of a fibrous protein or peptide and ThT, a fluorescent component due to autofluorescence of ThT, an apparent fluorescent component derived from the characteristics of a device for measuring a fluorescence lifetime, a fluorescent component derived from impurities contained in the sample, and the like.

When there is a noise component, the fitting step may be performed by obtaining G(t) represented by the following Mathematical Formula (4) including noise parameters (τₙ₊₁ to τₘ, Aₙ₊₁ to Aₘ) instead of G(t) represented by the above Mathematical Formula (1).

$$\begin{matrix}[ Math . 23 ] & \\ G ( t ) = {\sum }_{i = 1}^{m} [ {A}_{\dot{t}} exp ( - t / {τ}_{i} ) ] & ( 4 )\end{matrix}$$

[In Mathematical Formula (4), m represents a natural number of n+1 or more, n represents a natural number of 4 or more, Aᵢ represents the weighting factor of each exponential component, t represents a variable representing a time, and τᵢ represents the fluorescence lifetime value of each exponential component.]

### (Identifying Step)

The Identifying step is a step of identifying the structural polymorphism of a fibrous protein or peptide based on at least one or more of the fluorescence lifetime values τ₁ to τₙ and at least one or more of the weighting factors A₁ to Aₙ obtained in the fitting step (a fluorescence lifetime value and a weighting factor in an exponential component derived from autofluorescence of ThT are excluded).

Identifying the structural polymorphism of a fibrous protein or peptide may be, for example, estimating the structural polymorphism of a fibrous protein or peptide of interest, determining whether two structural polymorphisms of a fibrous protein or peptide of interest are identical, or the like.

In the identifying step, when the structural polymorphism of the fibrous protein or peptide of interest is estimated, for example, the identifying step may be performed by collating the fluorescence lifetime values τ₁ to τₙ and the weighting factors A₁ to Aₙ with a database storing fluorescence lifetime values τ₁ to τₙ and weighting factors A₁ to Aₙ associated with different structural polymorphisms of the fibrous protein or peptide, respectively. In the comparison between the fluorescence lifetime values τ₁ to τₙ and the weighting factors A₁ to Aₙ obtained in the fitting step and the fluorescence lifetime values τ₁ to Σₙ and the weighting factors A₁ to Aₙ stored in the database, if there is no significant difference in at least one or more of the fluorescence lifetime values τ₁ to τₙ and at least one or more of the weighting factors A₁ to Aₙ, it may be determined that both the structural polymorphisms are the same. That is, it can be determined that the structural polymorphism of the fibrous protein or peptide of interest is the same as the structural polymorphism with which the fluorescence lifetime values τ₁ to τₙ and the weighting factors A₁ to Aₙ stored in the database are associated. For example, when there is no significant difference between both τ₁ and A₁ obtained in the fitting step and a comparison object, both the structural polymorphisms may be determined to be the same. In the comparison between the fluorescence lifetime values τ₁ to τₙ and the weighting factors A₁ to Aₙ obtained in the fitting step and the fluorescence lifetime values τ₁ to τₙ and the weighting factors A₁ to Aₙ stored in the database, when there is a significant difference between any one of the fluorescence lifetime values τ₁ to τₙ and the weighting factors A₁ to Aₙ and the comparison object, both the structural polymorphisms may be determined to be different. For example, when there is a significant difference between A₁ obtained in the fitting step and the comparison object even if there is no significant difference between τ₁ obtained in the fitting step and the comparison object, both the structural polymorphisms may be determined to be different.

The database can be created, for example, as follows. First, the structural polymorphisms of various fibrous proteins or peptides are analyzed in advance by a cryo-electron microscope or the like to acquire data of the structural polymorphism of each fibrous protein or peptide. Thereafter, for the fibrous protein or peptide having the same structural polymorphism as that of each fibrous protein or peptide, the identifying method according to the present embodiment is performed to obtain the fluorescence lifetime values τ₁ to τₙ and the weighting factors A₁ to Aₙ. Subsequently, the data of the structural polymorphism of the fibrous protein or peptide is associated with the fluorescence lifetime values τ₁ to τₙ and the weighting factors A₁ to Aₙ.

In the identifying step, whether or not the structural polymorphisms of two fibrous proteins or peptides of interest are the same can be determined by comparing the fluorescence lifetime values τ₁ to τₙ and the weighting factors A₁ to Aₙ of the two.

The identifying step may be based on at least one or more of the fluorescence lifetime values τ₁ to τₙ and at least one or more of the weighting factors A₁ to Aₙ obtained in the fitting step (a fluorescence lifetime value and a weighting factor in an exponential component derived from autofluorescence of ThT are excluded), may be based on at least two or more, three or more, four or more, or all of the fluorescence lifetime values τ₁ to τₙ and at least two or more, three or more, four or more, or all of the weighting factors A₁ to Aₙ, or may be based on 50% or more, 60% or more, 70% or more, 80% or more, 90% or more, or 100% (all) of the fluorescence lifetime values τ₁ to τₙ and 50% or more, 60% or more, 70% or more, 80% or more, 90% or more, or 100% (all) of the weighting factors A₁ to Aₙ.

### [Device for Identifying Structural Polymorphism of Fibrous Protein or Peptide]

A device for identifying the structural polymorphism of a fibrous protein or peptide according to the present embodiment (hereinafter, also referred to as “device according to the present embodiment”) includes: a fluorescence lifetime measurement unit configured to obtain a fluorescence decay curve for a sample containing a fibrous protein or peptide and thioflavin T; a first calculation unit configured to perform exponential fitting of four or more components based on a function G(t) represented by the following Mathematical Formula (1) on a function F(t) of the fluorescence decay curve to obtain fluorescence lifetime values τ₁ to τₙ and weighting factors A₁ to Aₙ (n is a natural number of 4 or more) of the respective exponential components; and a second calculation unit configured to identify the structural polymorphism of the fibrous protein or peptide based on at least one or more values of the fluorescence lifetime values τᵢ to τₙ and at least one or more values of the weighting factors A₁ to Aₙ (a fluorescence lifetime value and a weighting factor in an exponential component derived from autofluorescence of ThT are excluded).

$$\begin{matrix}[ Math . 24 ] & \\ G ( t ) = {\sum }_{i = 1}^{n} [ {A}_{i} exp ( - t / {τ}_{i} ) ] & ( 1 )\end{matrix}$$

[In Mathematical Formula (1), n represents a natural number of 4 or more, Aᵢ represents the weighting factor of each exponential component, t represents a variable representing a time, and τᵢ represents the fluorescence lifetime value of each exponential component.]

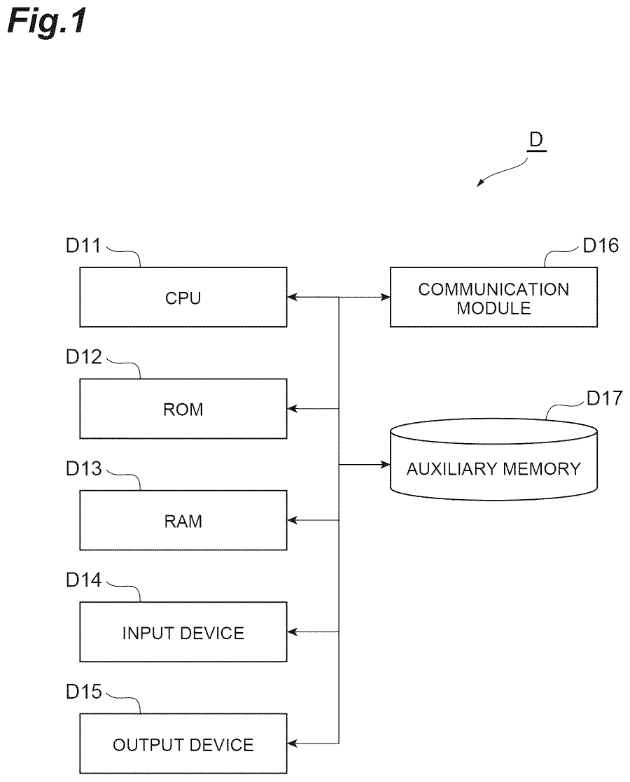

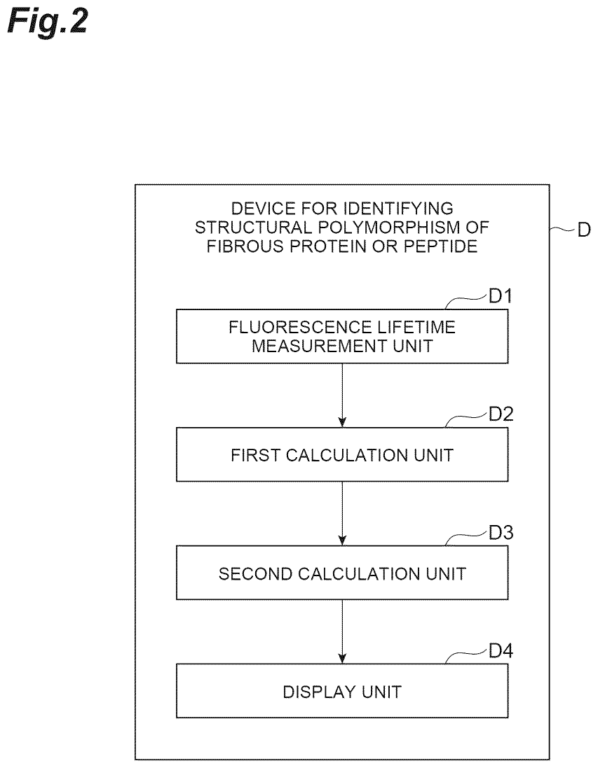

FIG. 1 is a schematic diagram showing the hardware configuration of a device D for identifying the structural polymorphism of a fibrous protein or peptide according to an embodiment, and FIG. 2 is a schematic diagram showing the functional configuration of the device D for identifying the structural polymorphism of a fibrous protein or peptide according to the embodiment.

As shown in FIG. 1, the device D for identifying the structural polymorphism of a fibrous protein or peptide is physically configured as a normal computer including a CPU D11, a main memory such as a ROM D12 and a RAM D13, an input device D14 such as a keyboard and a mouse, an output device D15 such as a display, a communication module D16 such as a network card for transmitting and receiving data to and from another device, for example, an imaging device C, and an auxiliary memory D17 such as a hard disk, and the like. Each function of the later-described device D for identifying the structural polymorphism of a fibrous protein or peptide is realized by loading a predetermined computer software on a hardware such as the CPU D11, the ROM D12, and the RAM D13, operating the input device D14, the output device D15, and the communication module D16 under the control of the CPU D11, and reading and writing data in the main memories D12 and D13 and the auxiliary memory D17.

As shown in FIG. 2, the device D for identifying the structural polymorphism of a fibrous protein or peptide according to the embodiment includes a fluorescence lifetime measurement unit D1, a first calculation unit D2, a second calculation unit D3, and a display unit D4 as functional components.

The fluorescence lifetime measurement unit D1 acquires fluorescence decay curve data obtained by a fluorescence lifetime measuring device (not shown). The first calculation unit D2 performs exponential fitting of four or more components based on a function G(t) represented by the above Mathematical Formula (1) on the fluorescence decay curve from the acquired fluorescence decay curve data to obtain fluorescence lifetime values τ₁ to τₙ and weighting factors A₁ to Aₙ (n is a natural number of 4 or more) of the respective exponential components. The second calculation unit D3 identifies the structural polymorphism of a fibrous protein or peptide based on the fluorescence lifetime values τ₁ to τₙ and the weighting factors A₁ to Aₙ (a fluorescence lifetime value and a weighting factor in an exponential component derived from autofluorescence of ThT are excluded). The display unit D4 displays the identified results. The fibrous protein or peptide is as described above, the aspect described in the measuring step can be applied to the fluorescence lifetime measurement unit, the aspect described in the fitting step can be applied to the first calculation unit, and the aspect described in the identifying step can be applied to the second calculation unit.

### [Program for Identifying Structural Polymorphism of Fibrous Protein or Peptide]

A program for identifying the structural polymorphism of a fibrous protein or peptide causes a computer to function as the fluorescence lifetime measurement unit D1, the first calculation unit D2, the second calculation unit D3, and the display unit D4 described above. By causing the computer to read the program for identifying the structural polymorphism of a fibrous protein or peptide, the computer operates as the device D for identifying the structural polymorphism of a fibrous protein or peptide. The program for identifying the structural polymorphism of a fibrous protein or peptide is provided, for example, by being recorded on a computer-readable recording medium. The recording medium may be a non-transitory recording medium. Examples of the recording medium include a recording medium such as a flexible disk, a CD, and a DVD, a recording medium such as a ROM, and a semiconductor memory. The fibrous protein or peptide is as described above, the aspect described in the measuring step can be applied to the fluorescence lifetime measurement unit, the aspect described in the fitting step can be applied to the first calculation unit, and the aspect described in the identifying step can be applied to the second calculation unit.

### EXAMPLES

Hereinafter, the present invention will be more specifically described based on Examples. However, the present invention is not limited to the following Examples.

### Test Example 1: Analysis of Fluorescence Lifetime of Thioflavin T in Amyloid β Fibers

### (1. Preparation of Measurement Samples)

Amyloid β protein (trade name: Amyloid β Protein Human 1-42, manufactured by Peptide Institute, Inc.) was dissolved in dimethyl sulfoxide to 5 mM (initial dissolution), and then diluted with 10 mM hydrochloric acid to a concentration of amyloid β protein of 100 μM. The obtained preparation solution of amyloid β protein was statically cultured at 37° C. using a shaking incubator (manufactured by AS ONE Corporation) to prepare a sample containing amyloid β fibers (hereinafter, also referred to as “amyloid β fiber sample”). Subsequently, 990 μL of a 5 μM ThT solution (solvent: 50 mmol/L glycine-sodium hydroxide solution, pH 9.0) was mixed with 10 μL of the amyloid β fiber sample to prepare measurement samples.

### (2. Acquisition of Fluorescence Decay Curves)

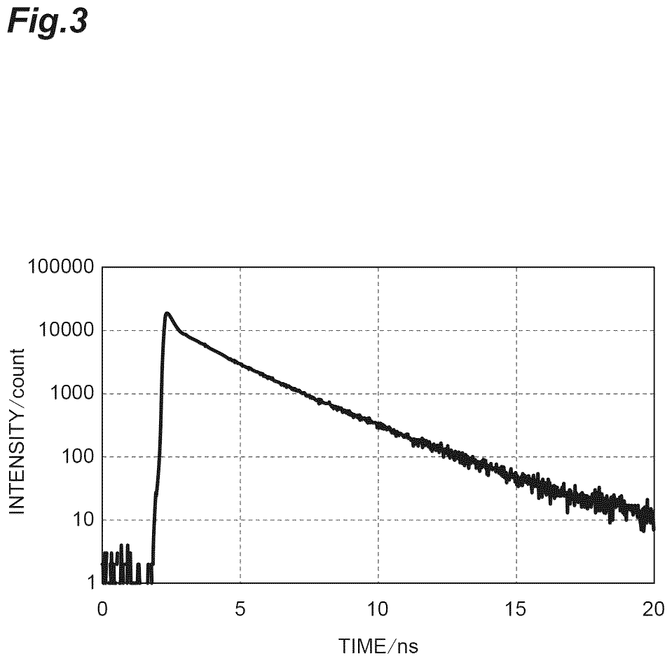

Each of the measurement samples obtained in (1. Preparation of Measurement Samples) was dispensed into a quartz cell. Thereafter, fluorescence decay curves were obtained at an excitation wavelength of 405 nm, an observation wavelength of 500 nm, and a time range of 20 ns (time resolution of 0.020 ns) using a fluorescence lifetime measuring device (manufactured by Hamamatsu Photonics K.K., trade name Quantaurus-Tau, light source: pulsed laser diode PLP-10 (manufactured by Hamamatsu Photonics K.K.)). The results are shown in FIG. 3.

### (3. Exponential Fitting of Four Components of Fluorescence Decay Curves)

Multicomponent exponential fitting was performed on the fluorescence decay curves F(t) obtained in (2. Acquisition of Fluorescence Decay Curves) by the following procedure according to Non-Patent Document (Desmond V. O'Connor et al, “Time-Correlated Single Photon Counting 1st Edition”, January 1984, pages 158-191) using a fluorescence lifetime value τ and a weighting factor A as variables.

First, a function G(t) represented by Mathematical Formula (1) was convolution-integrated according to Mathematical Formula (2) to obtain a function I(t). Next, I(t) described above was compared with the measured fluorescence decay curve F(t), and a combination of variables that minimize χ² in Mathematical Formula (3) to 1.2 or less was searched by a nonlinear least squares method so that both the functions matched best.

$$\begin{matrix}[ Math . 25 ] & \\ G ( t ) = {\sum }_{i = 1}^{n} [ {A}_{i} exp ( - t / {τ}_{i} ) ] & ( 1 )\end{matrix}$$

[In Mathematical Formula (1), n represents a natural number of 4 or more, Aᵢ represents the weighting factor of each exponential component, t represents a variable representing a time, and t; represents the fluorescence lifetime value of each exponential component.]

$$\begin{matrix}[ Math . 26 ] & \\ G ( t ) = {\sum }_{i = 1}^{n} [ {A}_{i} exp ( - t / {τ}_{i} ) ] & ( 2 )\end{matrix}$$

$$I ( t ) = \int E ( {t}^{′} ) G ( t - {t}^{′} ) {dt}^{′} + C = {\sum }_{{t}^{′} = 0}^{t} [ E ( {t}^{′} ) G ( t - {t}^{′} ) ] + C$$

[In Mathematical Formula (2), E(t) represents a device response function of a fluorescence lifetime measuring device, C represents a background, and t and t′ represent variables representing a time.]

$$\begin{matrix}[ Math . 27 ] & \\ {χ}^{2} = {\sum }_{j = {p}_{1}}^{{p}_{2}} \frac{{[ I ( {t}_{j} ) \to F ( {t}_{j} ) ]}^{2}}{I ( {t}_{j} )} / ( {p}_{2} - {p}_{1} + 1 ) & ( 3 )\end{matrix}$$

[In Mathematical Formula (3), tⱼ represents a variable representing a time, p₁ represents a start time of analysis, and p₂ represents an end time of analysis.]

By the multicomponent exponential fitting, it was found that the fluorescence decay curves obtained in the above (2. Acquisition of Fluorescence Decay Curves) require four exponential components C1 to C4, and τ₁ to τ₄ and A₁ to A₄ of the exponential components C1 to C4 were obtained. τ₁ to τ₄ of C1 to C4 are shown in Table 1.

TABLE 1

| | Fluorescence Lifetime |
|---|---|
| Component | Value |
| C1 | τ₁: 0.009 ns |
| C2 | τ₂: 0.23 ns |
| C3 | τ₃: 1.2 ns |
| C4 | τ₄: 2.5 ns |

τ₁ is the shortest fluorescence lifetime and is attributed to ThT not bound to amyloid β fibers. τ₂ to τ₄ are attributed to ThT bound to the amyloid β fibers. Therefore, in order to examine τ₂ to τ₄ in more detail, the behavior changes of the fluorescence intensities of the exponential components of C2 to C4 due to the increase in a ThT concentration were analyzed.

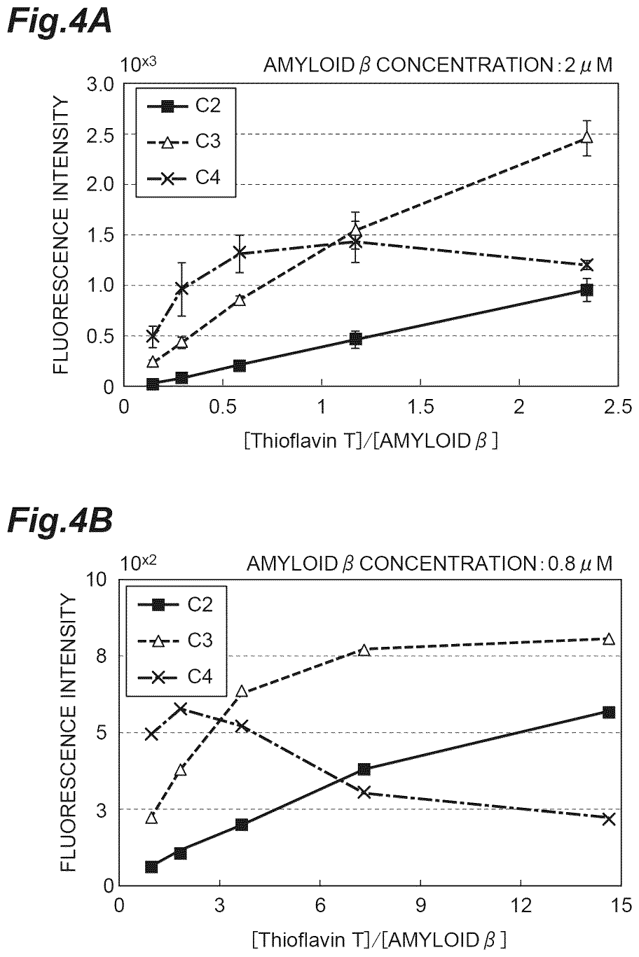

The fluorescence intensity of each exponential component when the concentration of amyloid β was set to 2 μM and the concentration of ThT with respect to the concentration of amyloid β ([ThT]/[amyloid β]) was set to 0.15, 0.29, 0.59, 1.2, or 2.3 was obtained by the following formula (5). Thereafter, each fluorescence intensity was plotted against [ThT]/[amyloid β]. Even under the condition where the concentration of ThT was higher, a graph showing a change in the fluorescence intensity depending on the concentration of ThT was obtained in the same manner as described above. At that time, the fluorescence intensity of each exponential component when the concentration of amyloid β was 0.8 μM and the concentration of ThT with respect to the concentration of amyloid β ([ThT]/[amyloid β]) was 0.91, 1.8, 3.7, 7.3, or 14.6 was also determined by the following formula (5). Thereafter, each fluorescence intensity was plotted against [ThT]/[amyloid β]. The results are shown in FIGS. 4A and 4B.

$$\begin{matrix}[ Math . 28 ] & \\ {F}_{i} = {τ}_{i} \times {A}_{i} & ( 5 )\end{matrix}$$

As shown in FIGS. 4A and 4B, behavior changes due to the ThT concentration were different between the C2 to C4 exponential components. In C4 determined by τ₄ having the highest fluorescence lifetime value, the fluorescence intensity was saturated first due to the increase in the ThT concentration, and then decreased.

Regarding amyloid β fibers and ThT, there is a report suggesting the presence of multiple types of ThT binding sites in the amyloid β fibers (Chun Wu et al., Biophys J. 2011 Mar. 2.; 100 (5): 1316-1324., Andrew Lockhart et al., J Biol Chem. 2005 Mar. 4; 280 (9): 7677-84). From such a report and the above results, it was considered that 12 to 14 are fluorescence lifetime values of ThT bound to different binding sites of the amyloid β fibers.

Therefore, by measuring each fluorescence lifetime value of ThT bound to the fibrous protein or peptide such as amyloid β fibers for each binding site, a slight difference in the structure of the fibrous protein or peptide can be measured as a difference between the fluorescence lifetime values, and the structural polymorphism of the fibrous protein or peptide can be identified.

### Test Example 2: Structure Identification Between Amyloid β (1-40) Fibers and Amyloid β (1-42) Fibers

### (1. Preparation of Measurement Samples)

Amyloid β protein (trade name: Amyloid β Protein Human 1-40 and Amyloid β Protein Human 1-42, manufactured by Peptide Institute, Inc.) was dissolved in 10 mM sodium hydroxide to 1 mg/mL, and then diluted 6-fold using a 50 mM phosphate buffer having a pH of 7.4. The obtained preparation solution of amyloid β protein was cultured by shaking (stirring: 800 rpm) at 37° C. using a shaking incubator (manufactured by AS ONE Corporation) to prepare a sample containing amyloid β fibers. Subsequently, an amyloid β fiber sample (final concentration: 2 μM), a ThT aqueous solution (final concentration: 4 μM), and a 50 mM phosphate buffer solution having a pH of 7.4 were mixed to prepare a measurement sample.

### (2. Acquisition of Fluorescence Decay Curves)

Fluorescence decay curves were measured in the same manner as in Test Example 1.

### (3. Exponential Fitting of Four Components of Fluorescence Decay Curves)

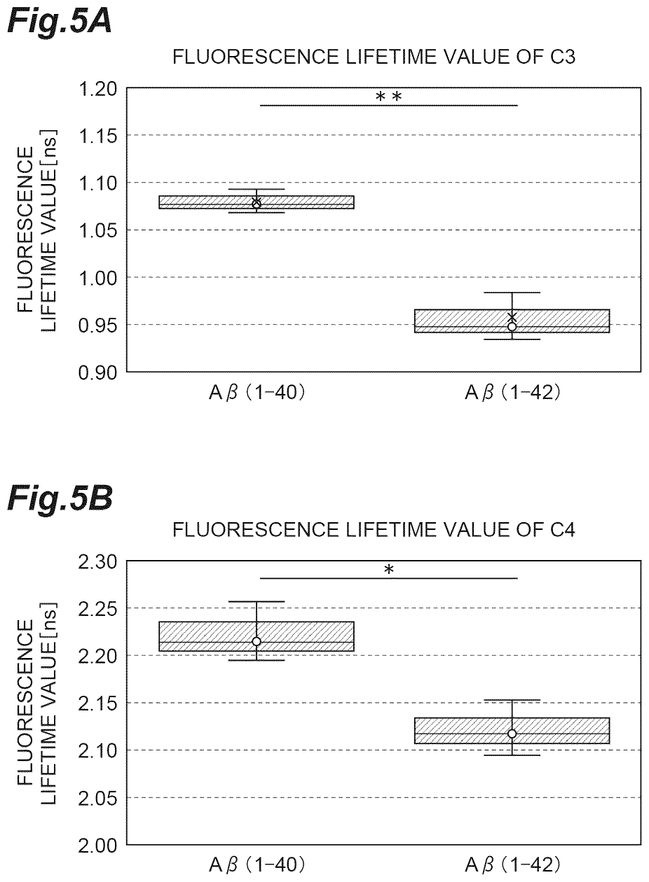

Exponential fitting of four components was performed on each obtained fluorescence decay curve F(t) in the same manner as in Test Example 1. τ₁ to τ₄ and A₁ to A₄ were obtained by performing the fitting. τ₁ to τ₄ and A₁ to A₄ are shown in Table 2. FIGS. 5A and 5B show the results of comparing the fluorescence lifetime value (13) of C3 or the fluorescence lifetime value (14) of C4 between the amyloid β (1-40) fibers and the amyloid β (1-42) fibers.

TABLE 2

| | | | | Difference between |
|---|---|---|---|---|
| | | Amyloid β | Amyloid β | Fluorescence Lifetime |
| | | (1-40) | (1-42) | Values |
| C2 | τ₂ | 0.28 ns | 0.27 ns | 0.01 |
| | A₂ | 1367 | 4628 | — |
| C3 | τ₃ | 1.08 ns | 0.96 ns | 0.12 |
| | A₃ | 1398 | 3097 | — |
| C4 | τ₄ | 2.22 ns | 2.12 ns | 0.10 |
| | A₄ | 687 | 867 | — |

As shown in Table 2 and FIGS. 5A and 5B, there was a significant difference in τ₃ and τ₄ between the amyloid β (1-40) fibers and the amyloid β (1-42) fibers. The amino acid sequences of the amyloid β (1-40) and amyloid β (1-42) fibers are different from each other, and the structures of the amyloid β fibers are also different from each other. By measuring a plurality of fluorescence lifetime values of the amyloid β fibers, it could be determined that the structures are different.

### Test Example 3: Identification of Structural Polymorphism of Amyloid β Fibers

Amyloid fibers having different structural polymorphisms are known to be formed when conditions for culturing amyloid β (1-42) are changed. In Test Example 3, amyloid β fibers having different structures were formed, and whether or not amyloids of proteins having the same amino acid sequence could be distinguished was examined.

### (1. Preparation of Measurement Samples)

Amyloid β (1-42) fiber samples were prepared in the same manner as in Test Example 1 except that the conditions were changed to those in the following Table 3. Subsequently, measurement samples were prepared using the respective prepared amyloid β fiber samples in the same manner as in Test Example 1 except that the compositions shown in the following Table 3 were adopted (Samples 1 to 3).

TABLE 3

| | Sample 1 | Sample 2 | Sample 3 |
|---|---|---|---|
| Fiber | Acid | Neutral | Neutral |
| Forming | (Stirring−) | (Stirring+) | (Stirring−) |
| Condition | | | |
| Initial | Concentration: | Concentration: 1 mg/mL | |
| Dissolution | 5 mM | Solvent: 10 mM NaOH | |
| | (22.57 mg/mL) | | |
| | Solvent: DMSO | | |
| Dilution | Concentration: | Concentration: 36.9 μM | |
| | 10 μM | Solvent: 50 mM Phosphate Buffer | |
| | Solvent: 10 mM | Solution | |
| | HCl | | |
| Culturing | Temperature: | Temperature: | Temperature: |
| Condition | 37° C. | 37° C. | 37° C. |
| | Stirring: None | Stirring: 800 rpm | Stirring: None |

| | |
|---|---|
| Measurement | Amyloid β Concentration: 2 μM, ThT Concentration: |
| Sample | 4 μM, 50 mM Phosphate Buffer Solution (pH 7.4) |

### (2. Acquisition of Fluorescence Decay Curves)

Fluorescence decay curves were measured in the same manner as in Test Example 1.

### (3. Exponential Fitting of Four Components of Fluorescence Decay Curves)

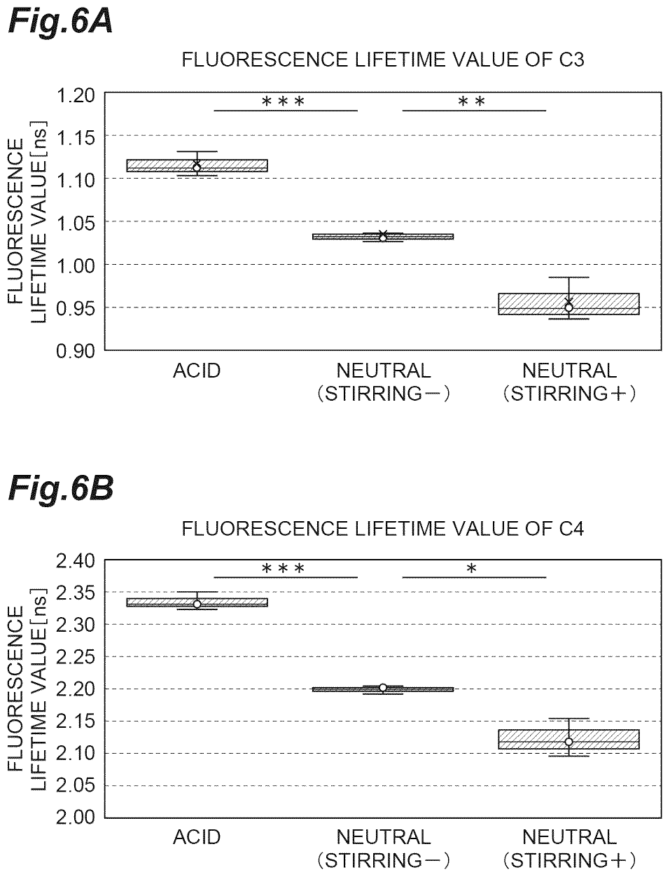

Exponential fitting of four components was performed on each obtained fluorescence decay curve F(t) in the same manner as in Test Example 1. τ₁ to τ₄ and A₁ to A₄ in Mathematical Formula (1) were obtained by performing the fitting. The results are shown in Table 4. FIGS. 6A and 6B show the results of comparing the fluorescence lifetime value (τ₃) of C3 or the fluorescence lifetime value (τ₄) of C4 between the samples. Furthermore, the ratio (%) of A₂ to the total value of A₁ to A₄ in each sample is shown in Table 5.

TABLE 4

| Compo- | | | | |
|---|---|---|---|---|
| nent | | Sample 1 | Sample 2 | Sample 3 |
| C1 | τ₁ | 0.005 ± 0.000 | 0.009 ± 0.011 | 0.002 ± 0.000 |
| | A₁ | 29074 ± 655 | 30292 ± 10609 | 29520 ± 224 |
| | τ₁ × A₁ | 132 ± 4 | 203 ± 165 | 60 ± 1 |
| C2 | τ₂ | 0.288 ± 0.004 | 0.266 ± 0.013 | 0.270 ± 0.005 |
| | A₂ | 965 ± 27 | 4628 ± 57 | 2131 ± 93 |
| | τ₂ × A₂ | 278 ± 6 | 1231 ± 63 | 576 ± 14 |
| C3 | τ₃ | 1.115 ± 0.014 | 0.955 ± 0.026 | 1.031 ± 0.005 |
| | A₃ | 1182 ± 23 | 3097 ± 8 | 1982 ± 67 |
| | τ₃ × A₃ | 1318 ± 20 | 2958 ± 89 | 2043 ± 60 |
| C4 | τ₄ | 2.333 ± 0.014 | 2.121 ± 0.029 | 2.196 ± 0.006 |
| | A₄ | 742 ± 25 | 867 ± 47 | 771 ± 9 |
| | τ₄ × A₄ | 1688 ± 55 | 1838 ± 79 | 1693 ± 23 |

TABLE 5

| | Sample 1 | Sample 2 | Sample 3 |
|---|---|---|---|
| Ratio of A₂ to A₁₊₂₊₃₊₄ | 3.0% | 11.9% | 6.2% |

As shown in Table 4 and FIGS. 6A and 6B, there was a significant difference between τ₃ and τ₄ obtained from each sample. As shown in Table 4, there was no significant difference in τ₂ obtained from each sample. That is, it is considered that similar binding sites exist in each sample. Meanwhile, as shown in Table 5, A₂ with respect to the total value of A₁ to A₄ in each sample was greatly different between the samples. From this, it is considered that the difference in the weighting factor indicates that the existence ratio of binding sites of ThT assumed from the value of τ₂ is different among the structural polymorphisms. From this result, it was found that the structural polymorphism of the amyloid β fibers having the same amino acid sequence can also be identified by measuring a plurality of fluorescence lifetime values and weighting factors.

From the results of Test Examples 1 to 3, it is considered that by measuring the fluorescence lifetime value for each binding site and measuring the weighting factor for each abundance of the binding site, a slight difference in the structure of the fibrous protein or peptide can be measured as a difference between each fluorescence lifetime value and each weighting factor.

### REFERENCE SIGNS LIST

D device for identifying structural polymorphism of fibrous protein or peptide
D1 fluorescence lifetime measurement unit
D2 first calculation unit
D3 second calculation unit
D4 display unit
D11 CPU
D12 ROM
D13 RAM
D14 input device
D15 output device
D16 communication module
D17 auxiliary memory

What is claimed is:

1. A method for identifying a structural polymorphism of a fibrous protein or peptide, the method comprising the steps of:
obtaining a fluorescence decay curve for a sample comprising a fibrous protein or peptide and thioflavin T;
performing exponential fitting of four or more components based on a function G(t) represented by the following Mathematical Formula (1) on a function F(t) of the fluorescence decay curve to obtain fluorescence lifetime values τ₁ to τₙ and weighting factors A₁ to Aₙ (n is a natural number of 4 or more) of the respective exponential components; and
identifying the structural polymorphism of the fibrous protein or peptide based on at least one or more values of the fluorescence lifetime values τ₁ to τₙ and at least one or more values of the weighting factors A₁ to Aₙ (a fluorescence lifetime value and a weighting factor in an exponential component derived from autofluorescence of thioflavin T are excluded),
$$\begin{matrix}[ Math . 1 ] & \\ G ( t ) = {\sum }_{i = 1}^{n} [ {A}_{i} exp ( - t / {τ}_{i} ) ] & ( 1 )\end{matrix}$$
wherein in Mathematical Formula (1), n represents a natural number of 4 or more, Aᵢ represents the weighting factor of each exponential component, t represents a variable representing a time, and τᵢ represents the fluorescence lifetime value of each exponential component.

2. The method according to claim 1, wherein the exponential fitting comprises:
convolution-integrating the function G(t) according to the following Mathematical Formula (2) to obtain a function I(t); and
comparing the function I(t) with the function F(t) of the fluorescence decay curve and searching a combination of variables minimizing χ² in the following Mathematical Formula (3) by a nonlinear least squares method,
$$\begin{matrix}[ Math . 2 ] & \\ I ( t ) = \int E ( {t}^{′} ) G ( t - {t}^{′} ) {dt}^{′} + C = {\sum }_{{t}^{′} = 0}^{t} [ E ( {t}^{′} ) G ( t - {t}^{′} ) ] + C & ( 2 )\end{matrix}$$
wherein in Mathematical Formula (2), E(t) represents a device response function of a fluorescence lifetime measuring device, C represents a background, and t and t′ represent variables representing a time,
$$\begin{matrix}[ Math . 3 ] & \\ {χ}^{2} = {\sum }_{j = {p}_{1}}^{{p}_{2}} \frac{{[ I ( {t}_{j} ) \to F ( {t}_{j} ) ]}^{2}}{I ( {t}_{j} )} / ( {p}_{2} - {p}_{1} + 1 ) & ( 3 )\end{matrix}$$
wherein in Mathematical Formula (3), tⱼ represents a variable representing a time, p₁ represents a start time of analysis, and p₂ represents an end time of analysis.

3. The method according to claim 1, wherein the identifying step is performed by collating the fluorescence lifetime values τ₁ to τₙ and the weighting factors A₁ to Aₙ with a database storing fluorescence lifetime values τ₁ to τₙ and weighting factors A₁ to Aₙ associated with different amyloid structural polymorphisms, respectively.

4. The method according to claim 1, wherein the exponential fitting is exponential fitting of four components.

5. The method according to claim 1, wherein a protein or peptide forming the fibrous protein or peptide is an amyloid-forming protein or peptide.

6. The method according to claim 5, wherein the amyloid-forming protein or peptide is one or more selected from the group consisting of amyloid β, α synuclein, transactive response DNA-binding protein-43, superoxide dismutase 1, prion protein, β2 microglobulin, immunoglobulin light chain protein, transthyretin, tau, and partial peptides thereof.

7. A device for identifying a structural polymorphism of a fibrous protein or peptide, the device comprising:
a fluorescence lifetime measurement unit configured to obtain a fluorescence decay curve for a sample comprising a fibrous protein or peptide and thioflavin T;
a first calculation unit configured to perform exponential fitting of four or more components based on a function G(t) represented by the following Mathematical Formula (1) on a function F(t) of the fluorescence decay curve to obtain fluorescence lifetime values τ₁ to τₙ and weighting factors A₁ to Aₙ (n is a natural number of 4 or more) of the respective exponential components; and
a second calculation unit configured to identify the structural polymorphism of the fibrous protein or peptide based on at least one or more values of the fluorescence lifetime values τ₁ to τₙ and at least one or more values of the weighting factors A₁ to Aₙ (a fluorescence lifetime value and a weighting factor in an exponential component derived from autofluorescence of thioflavin T are excluded),
$$\begin{matrix}[ Math . 4 ] & \\ G ( t ) = {\sum }_{i = 1}^{n} [ {A}_{i} exp ( - t / {τ}_{i} ) ] & ( 1 )\end{matrix}$$
wherein in Mathematical Formula (1), n represents a natural number of 4 or more, Aᵢ represents the weighting factor of each exponential component, t represents a variable representing a time, and τᵢ represents the fluorescence lifetime value of each exponential component.

8. The device according to claim 7, wherein the exponential fitting comprises:
convolution-integrating the function G(t) according to the following Mathematical Formula (2) to obtain a function I(t); and
comparing the function I(t) with the function F(t) of the fluorescence decay curve and searching a combination of variables minimizing χ² in the following Mathematical Formula (3) by a nonlinear least squares method,
$$\begin{matrix}[ Math . 5 ] & \\ I ( t ) = \int E ( {t}^{′} ) G ( t - {t}^{′} ) {dt}^{′} + C = {\sum }_{{t}^{′} = 0}^{t} [ E ( {t}^{′} ) G ( t - {t}^{′} ) ] + C & ( 2 )\end{matrix}$$
wherein in Mathematical Formula (2), E(t) represents a device response function of a fluorescence lifetime measuring device, C represents a background, and t and t′ represent variables representing a time,
$$\begin{matrix}[ Math . 6 ] & \\ {χ}^{2} = {\sum }_{j = {p}_{1}}^{{p}_{2}} \frac{{[ I ( {t}_{j} ) \to F ( {t}_{j} ) ]}^{2}}{I ( {t}_{j} )} / ( {p}_{2} - {p}_{1} + 1 ) & ( 3 )\end{matrix}$$
wherein in Mathematical Formula (3), tⱼ represents a variable representing a time, p₁ represents a start time of analysis, and p₂ represents an end time of analysis.

9. The device according to claim 7, wherein the identifying is performed by collating the fluorescence lifetime values τ₁ to τₙ and the weighting factors A₁ to Aₙ with a database storing fluorescence lifetime values τ₁ to τₙ and weighting factors A₁ to Aₙ associated with different amyloid structural polymorphisms, respectively.

10. The device according to claim 7, wherein the exponential fitting is exponential fitting of four components.

11. The device according to claim 7, wherein a protein or peptide forming the fibrous protein or peptide is an amyloid-forming protein or peptide.

12. The device according to claim 11, wherein the amyloid-forming protein or peptide is one or more selected from the group consisting of amyloid β, α synuclein, transactive response DNA-binding protein-43, superoxide dismutase 1, prion protein, β2 microglobulin, immunoglobulin light chain protein, transthyretin, tau, and partial peptides thereof.

13. A program for identifying a structural polymorphism of a fibrous protein or peptide,
the program for causing a computer to function as:
a fluorescence lifetime measurement unit configured to obtain a fluorescence decay curve for a sample comprising a fibrous protein or peptide and thioflavin T;
a first calculation unit configured to perform exponential fitting of four or more components based on a function G(t) represented by the following Mathematical Formula (1) on a function F(t) of the fluorescence decay curve to obtain fluorescence lifetime values τ₁ to τₙ and weighting factors A₁ to Aₙ (n is a natural number of 4 or more) of the respective exponential components; and
a second calculation unit configured to identify the structural polymorphism of the fibrous protein or peptide based on at least one or more values of the fluorescence lifetime values τ₁ to τₙ and at least one or more values of the weighting factors A₁ to Aₙ (a fluorescence lifetime value and a weighting factor in an exponential component derived from autofluorescence of thioflavin T are excluded),
$$\begin{matrix}[ Math . 7 ] & \\ G ( t ) = {\sum }_{i = 1}^{n} [ {A}_{i} exp ( - t / {τ}_{i} ) ] & ( 1 )\end{matrix}$$
wherein in Mathematical Formula (1), n represents a natural number of 4 or more, Aᵢ represents the weighting factor of each exponential component, t represents a variable representing a time, and τᵢ represents the fluorescence lifetime value of each exponential component.

14. The program according to claim 13, wherein the exponential fitting comprises:
convolution-integrating the function G(t) according to the following Mathematical Formula (2) to obtain a function I(t); and
comparing the function I(t) with the function F(t) of the fluorescence decay curve and searching a combination of variables minimizing χ² in the following Mathematical Formula (3) by a nonlinear least squares method,
$$\begin{matrix}[ Math . 8 ] & \\ I ( t ) = \int E ( {t}^{′} ) G ( t - {t}^{′} ) {dt}^{′} + C = {\sum }_{{t}^{′} = 0}^{t} [ E ( {t}^{′} ) G ( t - {t}^{′} ) ] + C & ( 2 )\end{matrix}$$
wherein in Mathematical Formula (2), E(t) represents a device response function of a fluorescence lifetime measuring device, C represents a background, and t and t′ represent variables representing a time,
$$\begin{matrix}[ Math . 9 ] & \\ {χ}^{2} = {\sum }_{j = {p}_{1}}^{{p}_{2}} \frac{{[ I ( {t}_{j} ) \to F ( {t}_{j} ) ]}^{2}}{I ( {t}_{j} )} / ( {p}_{2} - {p}_{1} + 1 ) & ( 3 )\end{matrix}$$
wherein in Mathematical Formula (3), tⱼ represents a variable representing a time, p₁ represents a start time of analysis, and p₂ represents an end time of analysis.

15. The program according to claim 13, wherein the identifying is performed by collating the fluorescence lifetime values τ₁ to τₙ and the weighting factors A₁ to Aₙ with a database storing fluorescence lifetime values τ₁ to τₙ and weighting factors A₁ to Aₙ associated with different amyloid structural polymorphisms, respectively.

16. The program according to claim 13, wherein the exponential fitting is exponential fitting of four components.

17. The program according to claim 13, wherein a protein or peptide forming the fibrous protein or peptide is an amyloid-forming protein or peptide.

18. The program according to claim 17, wherein the amyloid-forming protein or peptide is one or more selected from the group consisting of amyloid β, α synuclein, transactive response DNA-binding protein-43, superoxide dismutase 1, prion protein, β2 microglobulin, immunoglobulin light chain protein, transthyretin, tau, and partial peptides thereof.
